(19) United States
(12) Patent Application Publication
(10) Pub. No.: US 2026/0165809 A1
(43) Pub. Date: Jun. 18, 2026

(54) SYSTEMS AND METHODS FOR MEASURING FORCE APPLIED TO AN ENDOVASCULAR DEVICE

(71) Applicant: Rapid Medical Ltd., Yokneam (IL)

(72) Inventors: Yuval VAKNIN, Hanaton (IL); Giora KORNBLAU, Binyamina (IL); Aharon FRIEDMAN, Haifa (IL); Anatoly SHVEDIN, Haifa (IL)

(21) Appl. No.: 19/124,795

(22) PCT Filed: Oct. 26, 2023
(86) PCT No.: PCT/IB2023/060822

§ 371 Date: Apr. 25, 2025

Related U.S. Application Data
(60) Provisional application No. 63/381,288, filed on Oct. 27, 2022.
(60) Provisional application No. 63/447,842, filed on Feb. 23, 2023.

(51) Int. Cl. A61B 90/00; A61B 17/00; A61B 17/22

(52) U.S. Cl. CPC A61B 90/06; A61B 17/22; A61B 90/08; A61B 2017/00022; A61B 2017/00115; A61B 2017/00221; A61B 2017/00477; A61B 2090/064; A61B 2090/0807

(57) ABSTRACT

A force meter for detecting force applied to an endovascular device includes a housing, a passage in the housing configured to receive the endovascular device, and a sensor disposed in the housing to measure force from the endovascular device to the housing. The force meter may also include electronic components to process the force readings from the sensor, transmit the readings to an external receiver, and to indicate the force readings to a user.

### CROSS-REFERENCE TO RELATED APPLICATIONS AND INCORPORATION BY REFERENCE

The following applications are incorporated herein by reference in their entirety: U.S. Provisional Application No. 63/381,288 filed on Oct. 27, 2022; and U.S. Provisional Application No. 63/447,842, filed on Feb. 23, 2023.

### BACKGROUND

This disclosure relates to the field of endovascular medical devices. Specifically, this disclosure is related to systems and methods for measuring the force applied to endovascular devices intended to pass through a blood vessel of a patient to a target area inside the patient's body to perform a medical procedure.

An example of an endovascular treatment of the type relevant to this disclosure is the use of an endovascular device to treat narrowing, blockage, or hemorrhage in a blood vessel, including neurovascular, cardiovascular, and peripheral vasculatures. For instance, treatment of an acute stroke caused by a blockage of a blood vessel in the brain typically comprises either the intra-arterial administration of thrombolytic drugs such as recombinant tissue plasminogen activator (rtPA), mechanical removal of the blockage, or a combination of the two. These interventional treatments must occur within hours of the onset of symptoms. Both intra-arterial (IA) thrombolytic therapy and interventional thrombectomy involve accessing the blocked cerebral artery via endovascular techniques and devices.

Mechanical treatment involves the physical manipulation of the relevant structure to relieve the cause of the symptoms. For example, mechanical treatment of a blood clot involves the physical removal of the blood clot by various means, such as capturing the blood clot mechanically by use of a mesh, balloons, snares, or coils, with or without the addition of supporting techniques like the use of suction to remove the clot or stents to support the blood vessel. Another example of a mechanical treatment is the mechanical reshaping of blood vessels to improve blood flow, which is accomplished by the use of mechanical devices similar to those discussed above.

After this mechanical treatment is completed, the endovascular device must be retracted from the blood vessel. Movement of the endovascular device in the body, and especially during retraction, can cause damage to the blood vessels the endovascular device moves through because of the limited space between the endovascular device and the blood vessel walls. This issue is particularly relevant for endovascular devices that have physically captured material (e.g., a blood clot) for removal. In these cases, the capturing process usually involves a portion of the endovascular device having a larger section, such as an expanded snare or mesh. This larger section increases friction between the endovascular device and the blood vessel, which increases the risk of damage.

Current techniques to address this situation involve training the operator of the endovascular device and using imaging techniques to detect unwanted movement of blood vessels during the extraction process. Training ameliorates this problem to some extent, but relies on the skill of an individual operator, which can vary. Imaging also can be helpful but is reactive in nature because movement of a blood vessel during extraction would ideally be avoided entirely. Thus, there is a need for improved systems and methods to ensure excess force is not being applied during extraction of an endovascular device.

### BRIEF SUMMARY OF THE INVENTION

In an embodiment, a force meter for an endovascular device includes a housing; a hollow tube disposed in the housing with a bend, the hollow tube extending between two exterior sides of the housing and being configured to receive the endovascular device; at least one support point disposed in the housing and configured to contact the hollow tube to support the bend; and a sensor disposed in the housing configured to sense a force applied from the endovascular device.

In another embodiment, a system for measuring tensile force applied to an endovascular device includes the force meter of some embodiments of the invention, and an endovascular device disposed through the hollow tube of the force meter.

In another embodiment, a method of using a force meter to detect a tensile force applied to an endovascular device includes passing an endovascular device through the force meter of some embodiments of the invention; detecting a force applied from the endovascular device to a force sensor disposed in the housing; and processing the force to determine the tensile force applied to the endovascular device using electronic components operably connected to the force sensor.

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS/FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1 is a perspective view of an endovascular device according to an embodiment.

FIG. 2 is a perspective view of a force meter for an endovascular device according to an embodiment.

FIG. 3 is a side view of the force meter of FIG. 2 according to an embodiment.

FIG. 4 is a side view of the force meter of FIG. 2 with housing portions removed according to an embodiment.

FIG. 5 is a different side view of the force meter of FIG. 2 with housing portions removed according to an embodiment.

FIG. 6 is a side view of a force meter for an endovascular device with housing portions removed according to an embodiment.

FIG. 7 is a side view of a force meter for an endovascular device with housing portions removed according to an embodiment.

FIG. 8 is a block diagram of a system for measuring tensile force of an endovascular device according to an embodiment.

FIG. 9 is a flow diagram of a method for using a system for measuring tensile force of an endovascular device according to an embodiment.

In the drawings, like reference numbers generally indicate identical or similar elements. Additionally, generally, the left-most digit(s) of a reference number identifies the drawing in which the reference number first appears.

### DETAILED DESCRIPTION

Reference will now be made in detail to representative embodiments illustrated in the accompanying drawings. References to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such a feature, structure, or characteristic in connection with other embodiments whether or not explicitly described.

Removal of an endovascular device after performing a procedure provides a risk of damage to the relevant blood vessels due to the pulling of the endovascular device from the blood vessels. According to a first embodiment, a tensile force measurement apparatus for an endovascular device is formed from a housing with a hollow tube configured to allow the endovascular device to pass through the housing. The hollow tube includes a bent portion that includes at least one support portion configured to restrict movement of the endovascular device. A force meter is located inside the housing and is configured to measure a force imparted by the endovascular device to the force meter. Optionally, a passage is configured within the housing to receive the hollow tube, the passage being configured from one end of the housing to the other end of the housing.

Some benefits of these and other embodiments disclosed herein are an easy-to-use and accurate measure of the tensile force being applied to the endovascular device. The disclosed apparatuses and systems continuously measure the tensile force being applied and display the measured force in a simple manner, allowing the operator of the endovascular device to have immediate and clear feedback on the tensile force being applied. This reduces the risk of accidental damage to the relevant blood vessels and thereby improves patient outcomes.

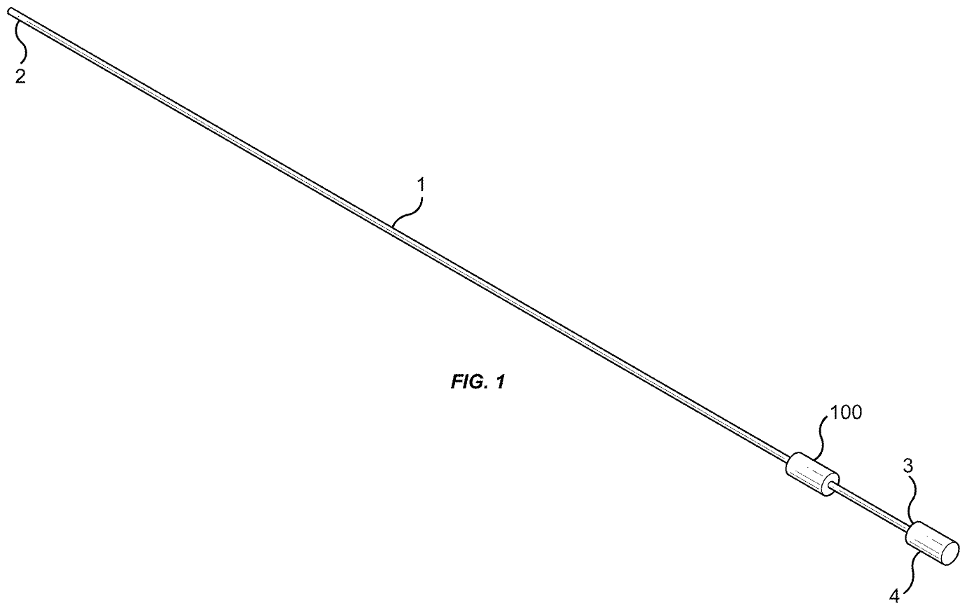

FIG. 1 shows a perspective view of an endovascular device 1 for use in performing endovascular procedures. Endovascular device 1 may be any type of endovascular device, including but not limited to, a device for accessing a location of interest or a device for performing a treatment at a location of interest, such as for example, a guidewire; a catheter, e.g., microcatheter, aspiration catheter; a stent; a balloon; a coil; a device for performing thrombectomy such as a stent retriever (including devices comprising a mesh or a snare); or an embolization assist device (such as devices comprising a mesh or a snare). Endovascular device 1 is generally sized and shaped to be at least partially inserted into a blood vessel of a patient. Endovascular device 1 includes a distal end 2 configured to be inserted into a blood vessel, and a proximal end 3 that is configured to remain outside of the patient. Optionally, a handle 4 is positioned at proximal end 3 for use by the operator to manipulate endovascular device 1. In some embodiments, portions of endovascular device, such as distal end 2, can include a mechanical treatment portion. Examples of devices comprising mechanical treatment portions include, but are not limited to, devices comprising an expandable mesh, devices comprising a snare, guidewires (e.g., steerable guide wires), balloon catheters, and stents. A force meter 100 is mounted to endovascular device 1 near proximal end 3 for measuring tensile force applied to endovascular device 1 during an endovascular procedure. Alternatively, endovascular device 1 is fitted through force meter 100 near proximal end 3 for measuring tensile force applied to endovascular device 1 during an endovascular procedure.

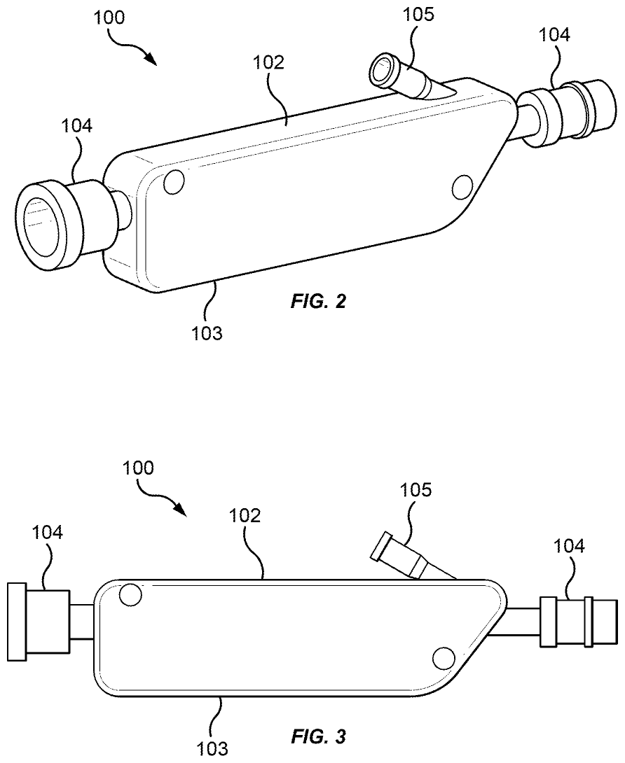

FIGS. 2-3 are a perspective and side view of an embodiment of force meter 100. A housing 102 forms the main body of force meter 100. Housing 102 of force meter 100 can be formed in any suitable shape, such as but not limited to, rectangular, square, hexagonal, tubular, trapezoidal, oval, round), can be tapered or non-tapered, can have a smooth surface or an uneven surface. According to one embodiment, as seen in FIGS. 2-3, housing 102 can be formed in a trapezoidal shape, with two opposite sides being longer than either of the other two sides. Housing 102 can be formed from any suitable material, including but not limited to, metal (e.g., stainless steel, nickel alloys, titanium, titanium alloys, or combinations thereof), plastic (e.g., thermoplastic such as polycarbonate, polypropylene or polyethylene), silicone, or composite materials. In some embodiments, housing 102 is shaped to be comfortably held by a user by hand. Housing 102 is configured to act as the supporting structure for the elements of force meter 100. According to some embodiments, housing 102 is at least partially hollow. According to a specific embodiment, housing 102 is configured to contain endovascular device 1 within and measure the tensile force on endovascular device 1.

Two valves 104 are found at the exterior of housing 102, with one valve 104 disposed at a first end of housing 102 and another valve 104 being disposed at a second end of housing 102. Valves 104 are fluidly connected to an interior of housing 102, as will be discussed in detail below. Valves 104 are configured to allow endovascular device 1 to pass through and into housing 102 and into a patient's body. According to one embodiment, valve 104 at the distal end of housing 102 is analogous to valve 104 at the proximal end of housing 102. According to one embodiment, valve 104 at the distal end of housing 102 is disparate from valve 104 at the proximal end of housing 102. According to a specific embodiment, valve 104 positioned at the distal end of housing 102 is configured as an adaptor valve for connecting housing 102 to a device for placement of endovascular device 1 in a patient's body, such as a catheter e.g., a guide catheter. According to a specific embodiment, valve 104 positioned at the proximal end of housing 102 is configured as a passage valve enabling passage of endovascular device 1 through housing 102 and into a patient's body. According to one embodiment, each of valves 104 may create a fluid-tight seal around endovascular device 1. According to a specific embodiment, valve 104 positioned at the proximal end of housing 102 is configured to create a fluid-tight seal around endovascular device 1.

Also shown is an introduction port 105, which is disposed near one of valves 104. According to one embodiment, introduction port 105 is disposed in proximity to valve 104 positioned at the distal end of housing 102. Introduction port 105 is configured to allow fluids containing, for example, saline, contrast agent or dye (e.g., for medical imaging such as, for example, X-ray, magnetic resonance imaging (MRI), computed tomography (CT), angiography, and ultrasound) or medication, into the patient's body via the fluid-tight space that contains endovascular device 1, as will be discussed in detail below. Introduction port 105 may have its own valve or other mechanism to allow for the introduction of fluids while preventing any fluid leaks. In some embodiments, there may be only one introduction port 105. In other embodiments there may not be an introduction port 105, or there may be more than one introduction port 105. It should be understood that in some embodiments, housing 102 can take the place of an existing catheter hub due to the inclusion of valves 104 and introduction port 105.

Shown in FIGS. 2-3 is a cover 103 on housing 102. Cover 103 can be fixed to the remainder of housing 102 by any suitable method, including but not limited to, mechanical fasters, adhesives, or welding. In some embodiments, cover 103 is intended to be removable to, for example, assemble or service force meter 100. In other embodiments, cover 103 is permanently fixed to housing 102 during manufacturing of force meter 100 by a suitable technique (as discussed above).

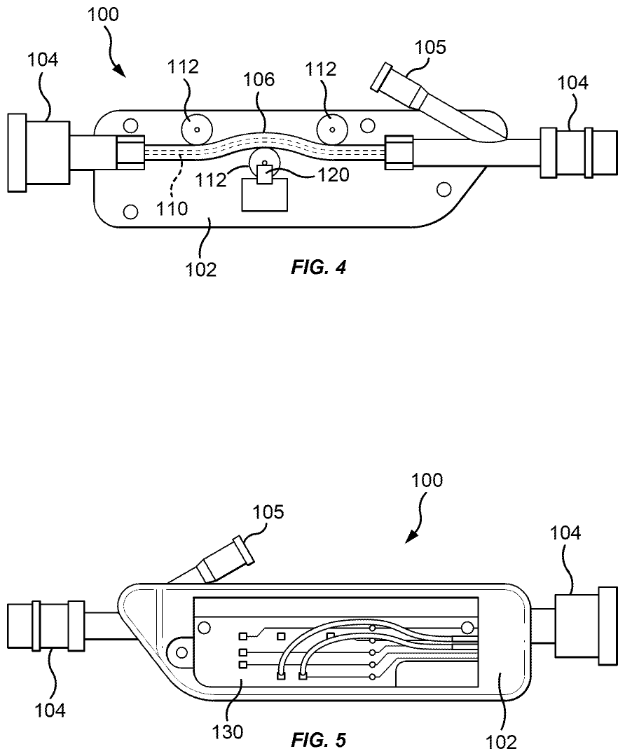

FIG. 4 is a side view of an embodiment of force meter 100 with cover 103 removed. This embodiment of force meter 100 shows a partially hollow housing 102. This means the interior of housing 102 is not completely hollow. Here, a completely hollow housing would be, for example, a hollow rectangular prism shape with solid walls that do not extend substantially into the hollow interior. According to one embodiment, the interior of housing 102 is at least partially solid, comprising openings or spaces to accommodate components of force meter 100 as needed. Typically, when housing 102 is partially hollow, a passage 106 is defined in housing 102 and connects the ends of housing 102 that support valves 104. Thus, passage 106 links two exterior sides of housing 102. Any two exterior sides of housing 102 can be linked by passage 106. In the embodiment of FIG. 4, the two shorter sides of housing 102 are linked by passage 106. Passage 106 is sized to accommodate endovascular device 1 as it passes through housing 102. Passage 106 is formed with a bend or deviation from the straight line between valves 104. This bend forces endovascular device 1 into a corresponding bent shape that, as will be discussed below, allows for the force measurement to take place.

Also shown in FIG. 4 is tube 110. Tube 110 is a hollow tube that fluidly connects valves 104 (and introduction port 105, if applicable). Tube 110 is present in both partially hollow and fully hollow housings 102. Typically, when passage 106 is present in housing 102 (usually in a partially hollow housing 102), tube 110 passes through passage 106. Thus, tube 110 ensures that any fluid is kept contained and separated from the other components found in housing 102. Tube 110 also enables fluids to be safely pressure pushed via force meter 100 (e.g., via introduction port 105, discussed below) into the patient's vasculature. That is, tube 110 can act as a fluid pathway that allows fluid to be passed through force meter 100. Accordingly, tube 110 enables to carry out the endovascular procedure without the force meter having an effect on the procedure itself. Tube 110 is sized to allow passage of endovascular device 1 therethrough. Tube 110 is also typically configured to be flexible and move with endovascular device 1 if endovascular device 1 flexes. According to one embodiment, the material for tube 110 is selected such that it will have high flexibility. According to one embodiment, the material for tube 110 is selected such that it will have low friction with respect to endovascular device 1. According to one embodiment, the material for tube 110 is selected such that it will not affect the baseline measurement of force meter 100. According to one embodiment, the material for tube 110 is selected such that the force measurement will reflect on the force generated by endovascular device 1 and not by tube 110. In some embodiments, tube 110 can be selected from a material including, but not limited to, polytetrafluoroethylene (“PTFE”) or Pebax. According to one embodiment, tube 110 includes a coating or coil inside to minimize friction between tube 110 and endovascular device 1. Such a coating or coil may be selected from, but not limited to, a PTFE coating or a stainless-steel coil.

Support points 112 are also shown in FIG. 4. Support points 112 are portions of passage 106 that are configured to act as stops or supports for endovascular device 1, also referred to as support structures. These stops physically prevent endovascular device 1 from moving beyond a certain point. Support points 112 can be separate inserts or elements embedded in passage 106, as shown in FIG. 4, or can also be formed as part of the walls that define passage 106. Alternatively, support points 112 can be separate inserts or elements embedded in housing 102, such as in situations wherein housing 102 is hollow and passage 106 is not present. In embodiments where support points 112 are separate inserts, these inserts can be selected to minimize friction between tube 110 and the surfaces of passage 106 or between tube 110 and the support points 112 themselves. For example, support points 112 can be patches of low-friction material such as PTFE, or could be devices like a roller or other rotating structure. Support points 112 included in a force meter 100 can be similar to one another or can be disparate from one another. It should be understood that the configuration of tube 110 and/or of passage 106 at least partially determines the placement and number of support points 112. According to one embodiment, the required bent configuration of tube 110 and/or of passage 106 at least partially determines the placement and number of support points 112. For example, support points 112 comprises a single support point disposed in housing 102. According to another embodiment, support points 112 comprises 2, 3, 4, 5 or more support point disposed in housing 102. According to a specific embodiment, support points 112 comprises 2 or 3 support points disposed in housing 102. Thus, in the embodiment of FIG. 4, there are three support points 112. This is because of the double-turn shape of the bend of passage 106 in FIG. 4. Other embodiments may have more or less support points 112 to accommodate and support the movement of tube 110 and endovascular device 1 therein.

A force sensor 120 is also shown in FIG. 4. Force sensor 120 is positioned inside housing 102 and configured to read a force imparted from the interaction between endovascular device 1 (through hollow tube 110) and force sensor 120. Force sensor 120 can be any suitable force sensor, including an analog sensor or a digital sensor. Exemplary sensors which may be used in accordance with some embodiments of the invention include, but are not limited to, load cell e.g., a piezoelectric sensor or a variable resistance sensor. Force sensor 120 is positioned inside housing 102. According to a specific embodiment, force sensor 120 is positioned inside passage 106. According to a specific embodiment, force sensor 120 is positioned in proximity to a support point 112. The combination of the bent portion of passage 106 and support points 112 guide endovascular device 1 to rest against force sensor 120. Note that in the embodiment of FIG. 4, where force sensor 120 is in direct contact with hollow tube 110, force sensor 120 is placed adjacent to one of support points 112 (in FIG. 4, the bottom center support point 112). This combination of features also means that any force applied along the length of endovascular device 1 will result in endovascular device 1 pressing against force sensor 120. The magnitude of the force applied to endovascular device 1 directly corresponds to the force that endovascular device 1 exerts on force sensor 120. Thus, force sensor 120 records a force that is correlated to the force applied to endovascular device 1.

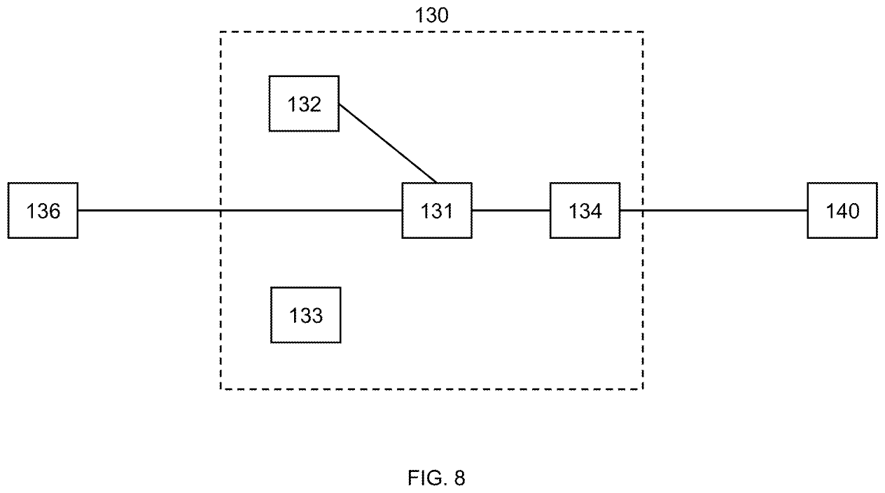

FIG. 5 is a different side view of the embodiment of FIG. 4 that shows the opposite side of an embodiment of force meter 100 with a portion of housing 102 removed. FIG. 5 shows a circuit board 130 disposed in housing 102. Circuit board 130 can contain some or all of the electronic components necessary for operation of force meter 100. In some embodiments, circuit board 130 includes processors and memory that are able to store and run the algorithms necessary to process the readings of force sensor 120. FIG. 8 is a system diagram of the electronic components of force meter 100. As seen in FIG. 8, circuit board 130 includes one or more processors 131 and memory 132. Circuit board 130 is operably connected to force sensor 120 to receive the force data from force sensor 120. This data is a force reading. This force reading, as explained above, is the force imparted by endovascular device 1 pressing against force sensor 120. These readings are converted to the force (e.g., tension force, also referred to as tensile force) applied along the length of endovascular device 1 by processor 131, which can use an experimentally determined equation or a data table to determine the corresponding force reading being applied to endovascular device 1. Circuit board 130 also includes a power source 133 for powering the electrical elements of force meter 100. Any suitable power source, such as but not limited to a battery, can be used for power source 133.

Also disposed on circuit board 130 is a transmitter 134. Transmitter 134 can be a wired or wireless communication transmitter. Transmitter 134 is operably connected to circuit board 130 and processor 131, is configured to receive the calculated force measurements and transmit those measurements to a suitable external receiver, as will be discussed below. In some embodiments, transmitter 134 can also include a receiving capability. Transmitter 134 can be any suitable data transmitter, including but not limited to, a universal serial bus (“USB”), Ethernet, Bluetooth, Wi-Fi, NFC or other wireless data protocol. Transmitter 134 may also include more than one transmit/receive capability, such as a USB capability and a Bluetooth capability. In embodiments with a wired communication capability, transmitter 134 can include a suitable external interface or socket, on housing 102, which can be sealed with a removable plug.

In some embodiments, the electronic components needed to collect and process the readings of force sensor 120 may be located outside of housing 102. Thus, processor 131 above may be located on a remote computing device that has an external receiver 140 that is in contact with force meter 130 via transmitter 134. It should be understood that there still may be a process disposed on circuit board 130 in housing 102 in these embodiments, but this processor may be programmed to receive sensor readings and transmit them using transmitter 134.

In some embodiments, a force indicator 136 is disposed in housing 102 and is operably connected to circuit board 130. Force indicator 136 can be used to indicate the magnitude of the tensile force being applied to endovascular device 1. FIG. 8 shows force indicator 136 to be a separate electrical component from circuit board 130. However, it should be understood that force indicator 136 could be physically disposed, at least in part, on circuit board 130. In some embodiments, force indicator 136 may be one or more lights visible from the exterior of housing 102. The lights may indicate force by changing color. For example, a green color may be displayed by force indicator 136 when the force readings are below a predetermined limit, a yellow color may be displayed by force indicator 136 when the force readings are approaching the predetermined limit, and a red color may be displayed by force indicator 136 when the force readings exceed the predetermined limit. Other light-based indications are possible, such as a flashing light to indicate exceeding the predetermined limit.

In some embodiments, force indicator 136 can include a vibrating element disposed in housing 102. This vibrating element can be used to create a vibration that can be felt by a user holding force meter 100. The vibrating element can be used to create various vibrations to indicate the tensile force being applied on endovascular device 1. For example, an intermittent vibration may indicate that the predetermined force limit is being approached, while a constant vibration may indicate the predetermined force limit has been exceeded.

In some embodiments, force indicator 136 can also include an audio element disposed in housing 102. This audio element can be used to create a sound that can be heard by a user holding force meter 100. The audio element can create various sounds to indicate different tensile forces being applied on endovascular device, similar to the vibration element discussed above. For example, intermittent sounds may indicate that the predetermined force limit is being approached, while a constant sound may indicate the predetermined force limit has been exceeded.

In some embodiments, force indicator 136 can include a display screen disposed on housing 102. The display screen can be any suitable type of display, such as but not limited to, an LCD display. The display screen can be used to display numerical force readings. The display screen can also display graphical indications of the force reading, such as but not limited to, using the numericals or using a graph, e.g., a bar graph or a line graph, and can display caution and warning icons when the predetermined force limit is being approached and exceeded, respectively.

Some embodiments of force indicator 136 include combinations of the options discussed above. Any combination is possible. For example, force indicator 136 may include both a vibrating element and lights. According to another embodiment, force indicator 136 may include both an audio element and lights. Other embodiments may include only the vibration element, only the audio element or only the lights.

As seen in FIG. 8, transmitter 134 can be operably connected to an external receiver 140. External receiver 140 can be any suitable computing device including, for example, a laptop, a desktop computer, a cellphone or a tablet. In some embodiments, external receiver 140 is a computing device that includes a display that can be used to show the force readings to the user of force meter 100. This can serve as an alternative indication of the force recorded by force meter 100, either in combination with or as a replacement for force indicator 136.

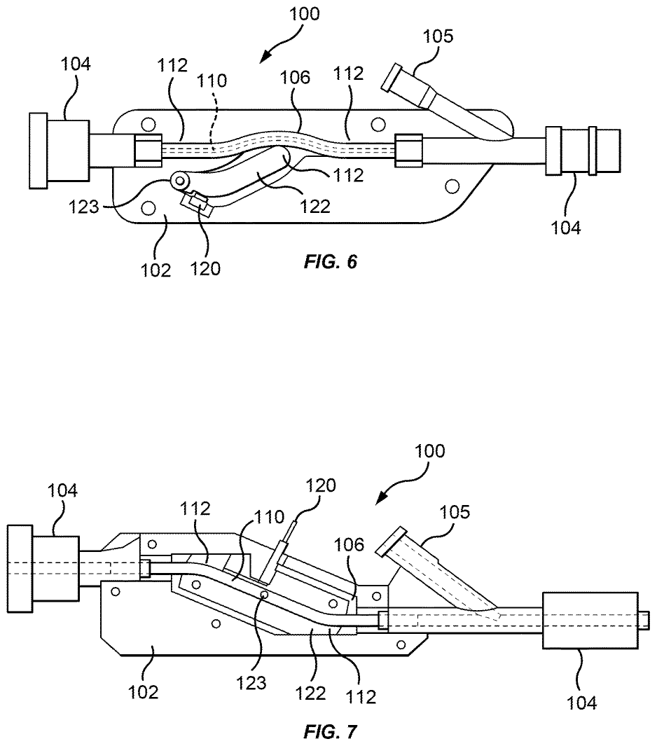

An embodiment of force meter 100 with a different measurement arrangement is shown in FIG. 6. The discussion above regarding housing 102, valves 104, passage 106, support points 112, and tube 110 applies equally here. This embodiment differs by placing force sensor 120 out of direct contact with hollow tube 110. Force sensor 120 is instead placed apart from tube 110 in housing 102. A lever 122 that has a pivot point 123 extends between force sensor 120 and tube 110, where lever 122 is in contact with endovascular device 1 (through hollow tube 110). Force applied to endovascular device 1 will be transmitted to lever 122, which in turn transmits the force to force sensor 120. Although this force is not a direct measurement of the tension force, it is directly correlated to the tension force, and thus these measurements can be used to calculate the tensile force by a suitable algorithm or look-up table determined by experiment. An advantage of the indirect measurement is that the arrangement of lever 122 and force sensor 120 allows for the force measured by force sensor 120 to be multiplied due to the leverage created by this arrangement. This has the benefit of increasing the force being measured by force sensor 120, which improves measurement accuracy because the magnitude of the forces in question is generally small, making those forces more difficult to measure accurately. This can also improve sensitivity of force sensor 120 because the forces being measured by force sensor 120 are larger. Accordingly, positioning of pivot point 123 can be used to multiply the force applied on force sensor 120 by moving pivot point 123 closer to force sensor 120 (as shown in FIG. 6). In either the direct contact embodiment of FIG. 4 or the lever embodiment of FIG. 6 (e.g., presenting indirect contact between hollow tube 110 and force sensor 120), an adjustment screw may be placed between force sensor 120 and the relevant structure (e.g., lever 122) to allow for adjustment of the sensitivity and readings of force sensor 120. Force sensor 120 is otherwise identical to force sensor 120 discussed above. The discussion of circuit board 130 and other electrical components above applies equally here.

FIG. 7 shows a different embodiment of force meter 100 that uses lever 122. In this embodiment, valves 104 are not arranged linearly because the bend in passage 106 is z-shaped such that valves 104 are not on the same level of housing 102. In some embodiments of FIG. 7, there are two fixed support points 112, which provide the z-shape of the bend of passage 106. According to some embodiments, support points 112 are constructed as part of lever 122. According to one embodiment. lever 122 is disposed in passage 106 and is formed such that hollow tube 110 and endovascular device 1 pass though lever 122. Thus, according to one embodiment, a passage is formed in lever 122 allowing the hollow tube to pass through a well-defined position of lever 122. According to one embodiment, pivot point 123 is positioned near the center of lever 122. Alternatively, the positioning of pivot point 123 may be altered to adjust the force applied on force sensor 120, as discussed above. It will be appreciated that force sensor 120 may be placed above or below lever 122, as long as it comes in contact, i.e., direct contact or indirect contact (e.g., via an adjustment screw), with lever 122, as discussed above. A force applied along the length of endovascular device 1 will result in rotation of lever 122 about pivot point 123. For example, a tensile force applied from the right to the left in FIG. 7 would rotate lever 122 counter-clockwise because of the shape of passage 106 and lever 122. Lever 122 contacts force sensor 120, which measures the force as discussed above. This arrangement results in a multiplication of the force on force sensor 120 for the same reasons discussed above. The remaining discussion above with respect to the other components for force meter 100 apply equally here.

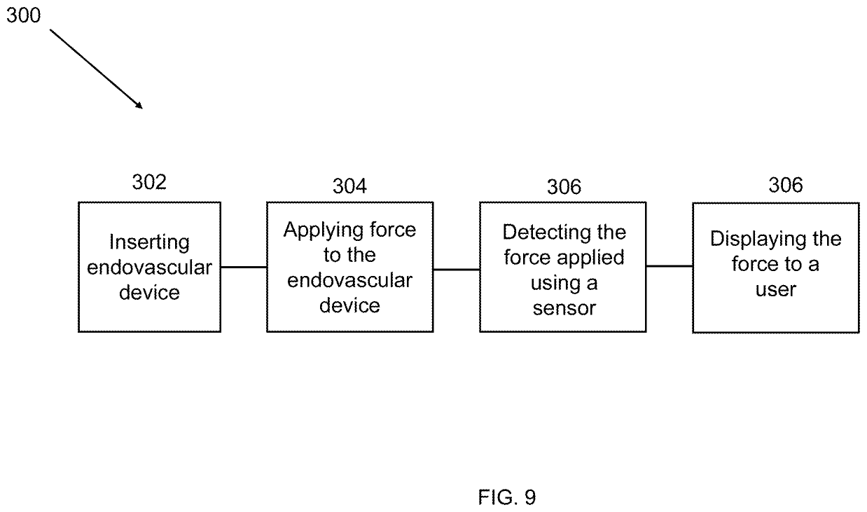

As shown in FIG. 9, a method 300 of using force meter 100 begins at a step 302 by inserting endovascular device 1 into force meter 100. As discussed above, valves 104 can be used to seal the interior of force meter 100 to avoid flow of fluids. A step 304 involves applying a tensile force to endovascular device 1. At step 306, force sensor 120 detects the force applied, and processor 131 processes that force to the corresponding tensile force applied to endovascular device 1. At step 308, the resulting force is displayed to the user, either by indicator 136 or by transmission via transmitter 134 to external receiver 140.

Exemplary embodiments of the invention are further provided below.

### EXAMPLE 1

A force meter for an endovascular device, comprising: a housing; a hollow tube disposed in the housing formed with a bend, the hollow tube extending between two exterior sides of the housing and being configured to receive the endovascular device; at least one support point disposed in the housing and configured to contact the hollow tube to support the bend; and a sensor disposed in the housing configured to sense a force applied from the endovascular device.

### EXAMPLE 2

The force meter of example 1, further comprising electronic components disposed in the housing and configured to receive a reading from the sensor and to determine a force applied to the endovascular device based on the reading.

### EXAMPLE 3

The force meter of example 2, further comprising a transmitter disposed in the housing, the transmitter operably connected to the electronic components, wherein the electronic components are configured to use the transmitter to transmit at least one of the reading from the sensor or of the force applied to the endovascular device to an external receiver.

### EXAMPLE 4

The force meter of example 3, wherein the transmitter comprises a wireless transmitter.

### EXAMPLE 5

The force meter of any one of examples 2-4, further comprising at least one of a processor and a memory disposed in the housing and operably connected to the electronic components.

### EXAMPLE 6

The force meter of any one of examples 2-5, further comprising an indicator disposed in the housing, the indicator operably connected to the electronic components, wherein the electronic components are configured to use the indicator to indicate the force applied to the endovascular device.

### EXAMPLE 7

The force meter of example 6, wherein the indicator comprises a light disposed in the housing and visible from an exterior of the housing.

### EXAMPLE 8

The force meter of example 6, wherein the indicator comprises a vibrating element disposed in the housing.

### EXAMPLE 9

The force meter of example 6, wherein the indicator comprises an audio element disposed in the housing.

### EXAMPLE 10

The force meter of any one of examples 1-9, further comprising a passage in the housing connecting two exterior sides of the housing and configured to receive the hollow tube.

### EXAMPLE 11

The force meter of example 10, wherein the at least one support point is disposed in the passage and configured to contact the hollow tube.

### EXAMPLE 12

The force meter of any one of examples 1-11, further comprising a lever disposed in the housing, the lever fixed to a pivot and positioned such that one portion of the lever is in contact with the hollow tube and a second portion of the lever is in contact with the force sensor, the lever configured to transmit a force applied from the endovascular device to the sensor.

### EXAMPLE 13

The force meter of any one of examples 1-11, wherein the sensor is positioned directly in contact with the hollow tube.

### EXAMPLE 14

The force meter of any one of examples 1-11, further comprising a lever disposed in the housing and fixed to a pivot, the lever containing an opening to allow the hollow tube to pass through the lever, the lever being further configured to transmit a force applied from the endovascular device to the sensor.

### EXAMPLE 15

The force meter of any one of examples 1-14, wherein the hollow tube is a flexible hollow tube.

### EXAMPLE 16

The force meter of any one of examples 1-15, further comprising a valve disposed on an exterior side of the housing, wherein the hollow tube is connected to the valve at the exterior side of the housing, the valve being configured to receive the endovascular device and fluids.

### EXAMPLE 17

The force meter of example 16, further comprising an introduction port disposed in proximity to the at least one valve and configured to allow introduction of fluids.

### EXAMPLE 18

A system for measuring tensile force applied to an endovascular device, comprising: the force meter of any one of examples 1-17; and an endovascular device disposed through the hollow tube of the force meter.

### EXAMPLE 19

The system of example 18, wherein the endovascular device comprises at least one of a clot retrieval device, a device comprising a snare, a device comprising a coil, a device comprising an expandable mesh, a guidewire, a balloon catheter, and a stent.

### EXAMPLE 20

The system of examples 18 or 19, wherein the tensile force applied to the endovascular device is at least partially impacted when the endovascular device is retracted through a blood vessel.

### EXAMPLE 21

A method of using a force meter to detect a tensile force applied to an endovascular device, comprising: passing an endovascular device through the force meter of any one of claims 1-17; detecting a force applied from the endovascular device to a force sensor disposed in the housing; and processing the force to determine the tensile force applied to the endovascular device using electronic components operably connected to the force sensor.

### EXAMPLE 22

The method of example 21, further comprising transmitting at least one of the force applied to the force sensor or the tensile force to an external receiver using a transmitter disposed in the housing.

### EXAMPLE 23

The method of any one of examples 21-22, further comprising indicating the tensile force applied to the endovascular device using an indicator disposed in the housing.

### EXAMPLE 24

The example of claim 23, wherein indicating the tensile force comprises at least one of illuminating a light disposed in the housing and visible from an exterior of the housing, vibrating a vibrating element disposed in the housing and using an audio element disposed in the housing.

### EXAMPLE 25

The method of any one of examples 210-24, wherein detecting the force comprises detecting movement of a lever disposed in the housing, the lever fixed to a pivot and positioned such that one portion of the lever is in contact with the hollow tube and a second portion of the lever is in contact with the force sensor, the lever configured to transmit a force applied from the endovascular device to the sensor.

### EXAMPLE 26

The method of any one of examples 21-24, wherein detecting the force comprises directly sensing the force from the hollow tube by the sensor.

### EXAMPLE 27

The method of any one of examples 21-244 wherein detecting the force further comprises detecting movement of a lever disposed in the housing, the lever fixed to a pivot and positioned such that one portion of the lever is in contact with the hollow tube and a second portion of the lever is in contact with the force sensor, the lever configured to transmit a force applied from the endovascular device to the sensor.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way. Moreover, the examples described above do not limit the present disclosure to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

The use of the modifiers “approximately” or “about” in this disclosure are intended to indicate that the relevant element is subject to variation by a tolerance range. Unless otherwise defined, the use of these modifiers with respect to a unit of measure means a tolerance of plus or minus ten percent of the unit of measure. The use of these modifiers with respect to a description such as a shape is intended to allow for variations of that shape due to tolerance issues as would be understood to occur in the art in general.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination. The breadth and scope of the present invention should not be limited by any of the above-described exemplary embodiments, but should be defined only in accordance with the following claims and their equivalents.

1. A force meter for an endovascular device, comprising:
a housing;
a hollow tube disposed in the housing formed with a bend, the hollow tube extending between two exterior sides of the housing and being configured to receive the endovascular device;
at least one support point disposed in the housing and configured to contact the hollow tube to support the bend; and
a sensor disposed in the housing configured to sense a force applied from the endovascular device.

2. The force meter of claim 1, further comprising electronic components disposed in the housing and configured to receive a reading from the sensor and to determine a force applied to the endovascular device based on the reading.

3. The force meter of claim 2, further comprising a transmitter disposed in the housing, the transmitter operably connected to the electronic components, wherein the electronic components are configured to use the transmitter to transmit at least one of the reading from the sensor or of the force applied to the endovascular device to an external receiver.

4. (canceled)

5. (canceled)

6. The force meter of claim 2, further comprising an indicator disposed in the housing, the indicator operably connected to the electronic components, wherein the electronic components are configured to use the indicator to indicate the force applied to the endovascular device.

7. The force meter of claim 6, wherein the indicator comprises at least one of a light disposed in the housing and visible from an exterior of the housing, a vibrating element disposed in the housing, or an audio element disposed in the housing.

8. (canceled)

9. (canceled)

10. The force meter of claim 1, further comprising a passage in the housing connecting two exterior sides of the housing and configured to receive the hollow tube.

11. The force meter of claim 10, wherein the at least one support point is disposed in the passage and configured to contact the hollow tube.

12. The force meter of claim 1, further comprising a lever disposed in the housing, the lever fixed to a pivot and positioned such that one portion of the lever is in contact with the hollow tube and a second portion of the lever is in contact with the force sensor, the lever configured to transmit a force applied from the endovascular device to the sensor.

13. The force meter of claim 1, wherein the sensor is positioned directly in contact with the hollow tube.

14. The force meter of claim 1, further comprising a lever disposed in the housing and fixed to a pivot, the lever containing an opening to allow the hollow tube to pass through the lever, the lever being further configured to transmit a force applied from the endovascular device to the sensor.

15. The force meter of claim 1, wherein the hollow tube is a flexible hollow tube.

16. The force meter of claim 1, further comprising a valve disposed on an exterior side of the housing, wherein the hollow tube is connected to the valve at the exterior side of the housing, the valve being configured to receive the endovascular device and fluids.

17. The force meter of claim 16, further comprising an introduction port disposed in proximity to the at least one valve and configured to allow introduction of fluids.

18. A system for measuring tensile force applied to an endovascular device, comprising:
the force meter of claim 1; and
an endovascular device disposed through the hollow tube of the force meter.

19. (canceled)

20. (canceled)

21. A method of using a force meter to detect a tensile force applied to an endovascular device, comprising:
passing an endovascular device through a hollow tube of the force meter of claim 1;
detecting a force applied from the endovascular device to a force sensor disposed in the housing; and
processing the force to determine the tensile force applied to the endovascular device using electronic components operably connected to the force sensor.

22. The method of claim 21, further comprising transmitting at least one of the force applied to the force sensor or the tensile force to an external receiver using a transmitter disposed in the housing.

23. The method of claim 1, further comprising indicating the tensile force applied to the endovascular device using an indicator disposed in the housing.

24. (canceled)

25. The method of claim 1, wherein detecting the force further comprises detecting movement of a lever disposed in the housing, the lever fixed to a pivot and positioned such that one portion of the lever is in contact with the hollow tube and a second portion of the lever is in contact with the force sensor, the lever configured to transmit a force applied from the endovascular device to the sensor.

26. The method of claim 1, wherein detecting the force further comprises directly sensing the force from the hollow tube by the sensor.

27. The method of claim 1, wherein detecting the force further comprises detecting movement a lever disposed in the housing and fixed to a pivot, an opening formed in the lever to allow the hollow tube to pass through the lever, the lever configured to transmit a force applied from the endovascular device to the sensor.
